(19) United States
(12) Patent Application Publication
(10) Pub. No.: US 2020/0051698 A1
(43) Pub. Date: Feb. 13, 2020

(54) PRECISION CLINICAL DECISION SUPPORT WITH DATA DRIVEN APPROACH ON MULTIPLE MEDICAL KNOWLEDGE MODULES

(71) Applicant: KONINKLIJKE PHILIPS N.V., EINDHOVEN (NL)

(72) Inventors: Tak Ming Chan, Shanghai (CN); Qin Zhu, Shanghai (CN); Chun Qi Shi, Shanghai (CN); Choo Chiap Chiau, Shanghai (CN)

(21) Appl. No.: 16/342,452

(22) PCT Filed: Oct. 18, 2017
(86) PCT No.: PCT/EP2017/076552

§ 371 Date: Apr. 16, 2019

(30) Foreign Application Priority Data
Nov. 3, 2016 (CN) PCT/CN2016/104462
Jan. 11, 2017 (EP) 17151018.3

(51) Int. Cl. G16H 50/70; G16H 10/60; G06F 16/907; G06F 16/906

(52) U.S. Cl. CPC G16H 50/70; G06F 16/906; G06F 16/907; G16H 10/60

(57) ABSTRACT

An electronic clinical decision support (CDS) device executes (54) clinical decision rules (8) using a computer (10, 12) to generate predicted values of clinical conclusions (58) for a current patient based on values for the current patient of preconditions of the clinical decision rules (52). The rules are also executed (36) to generate predicted values of the clinical conclusions (38) for past patients based on values for the past patients of the preconditions (32) retrieved from an Electronic Medical Record (EMR) (20). Rule summary scores (42) are generated (40) based on comparisons of “ground truth” values of the clinical conclusions (34) for the past patients retrieved from the EMR with the predicted values of the clinical conclusions for the past patients. A display (14) shows the predicted values of the clinical conclusions and the corresponding applied rules for the current patient ranked at least in part by the rule summary scores.

### FIELD

The following relates generally to the electronic clinical decision support (CDS) device arts, rules-based electronic CDS device arts, medical care delivery arts, and the like.

### BACKGROUND

An electronic clinical decision support (CDS) device comprises an electronic data processing device, e.g. a computer, which is programmed to provide clinical recommendations on the basis of patient-specific information. In rules-based electronic CDS devices, a set of clinical decision rules are employed for this purpose. Each clinical decision rule is typically formulated as a set of preconditions and a clinical conclusion, and can be heuristically written as:
If <preconditions met by patient> then present <clinical conclusion>
   The clinical decision rule is executed using the values of the preconditions to generate the value of the clinical conclusion. Using an electronic CDS device in a hospital, clinic, or other medical facility advantageously provides context-sensitive access to medical knowledge that might otherwise be unavailable to physicians or other medical staff of the medical facility. The electronic CDS device also enhances uniformity in medical diagnoses and treatment amongst physicians of the medical facility. Moreover, if the medical facility employs an Electronic Medical Record (EMR) (sometimes referred to as an Electronic Health Record or the like), then the electronic CDS device may be synergistically integrated with the EMR so that patient information on preconditions can be automatically imported to the electronic CDS device from the EMR. This ensures that available patient data are leveraged in making the clinical assessment.

The efficacy of a rules-based electronic CDS device depends on the quantity and quality of the implemented clinical decision rules. These rules may initially be formulated by a committee of skilled medical experts. However, relying entirely on such an anecdotal approach is not ideal. Rather, the proposed rules should be further developed and validated by way of clinical studies under the direction of medical researchers and preferably performed on a large patient sample with sufficient diversity (or vice versa, sufficient specificity on a targeted population) to encompass the various demographic categories and other classifications of patients that are expected to be diagnosed using the electronic CDS device. Rollout of an electronic CDS device product may also include obtaining approval of the underlying clinical decision rules from qualified medical associations, and/or obtaining approval from the Food and Drug Association (FDA, in the United States) or other governing regulatory agency, and/or other types of official approval or certification. The process of developing and validating clinical decision rules and obtaining appropriate approvals/certifications can be lengthy and expensive, and is likely to be carried out by large medical institutions, health care corporations, or other entities with extensive resources.

The following discloses new and improved systems, device, and methods.

### SUMMARY

In one disclosed aspect, an electronic clinical decision support (CDS) device is disclosed. A database stores clinical decision rules. Each clinical decision rule comprises a set of preconditions and is executable to generate a predicted value of a clinical conclusion that is dependent on values of the set of preconditions of the clinical decision rule. A computer hosts, or is connected by a data network with, an electronic medical record (EMR) of a past patient. The EMR contains determined values of preconditions and determined values of a clinical conclusion. The computer is programmed to perform clinical decision support for a current patient by obtaining values of the preconditions of the clinical decision rules for the current patient and executing the clinical decision rules using the obtained values of the preconditions for the current patient to generate predicted values of the clinical conclusions for the current patient. The computer is further programmed to perform a rules ranking process including: retrieving, from the EMR, the determined values of the preconditions of the clinical decision rules for past patients and the determined values of the clinical conclusions of the clinical decision rules for the past patients; for each past patient, executing the clinical decision rules using the values of the preconditions obtained for the past patients to generate predicted values of the clinical conclusions for the past patients; and generating rule summary scores for the clinical decision rules based on comparisons of the retrieved determined values of the clinical conclusions for the past patients with the predicted values of the clinical conclusions for the past patients.

In some embodiments, the CDS device further includes a display operatively connected with the computer, which is configured to display at least a sub-set of the predicted values of clinical conclusions and the corresponding applied rules for the current patient ranked at least in part by the rule summary scores of the clinical decision rules executed to generate the predicted values of clinical conclusions for the current patient. In some embodiments the display is configured to include an indication of any predicted values of clinical conclusions for the current patient that are produced by clinical decision rules whose rule summary scores indicate reliability of the clinical decision rule is below a threshold reliability.

In another disclosed aspect, a non-transitory storage medium stores a database of clinical decision rules, each clinical decision rule comprising a set of preconditions and being executable to generate a predicted value of a clinical conclusion that is dependent on values of the set of preconditions of the clinical decision rule, and instructions readable and executable by a computer to perform an electronic CDS method. The electronic CDS method includes: obtaining values of the preconditions of the clinical decision rules for a current patient; executing the clinical decision rules to generate predicted values of clinical conclusions for the current patient based on the obtained values for the current patient of preconditions of the clinical decision rules; retrieving, from an EMR, values of the preconditions of the clinical decision rules for past patients and values of the clinical conclusions of the clinical decision rules for the past patients; for each past patient, executing the clinical decision rules using the values of the preconditions retrieved from the EMR for the past patient to generate predicted values of the clinical conclusions for the past patients;

generating rule summary scores for the clinical decision rules based on comparisons of the retrieved values of the clinical conclusions for the past patients with the predicted values of the clinical conclusions for the past patients to prioritize the clinical decision rules or the predicted values generated by the clinical conclusion for the current patient; and displaying on a display a ranking of predicted values of clinical conclusions as well as the corresponding rules for the current patient ranked at least in part by the rule summary scores of the clinical decision rules executed to generate the predicted values of clinical conclusions for the current patient. In some embodiments, generating the rule summary scores includes clustering the clinical decision rules into groups of rules and generating a rule summary score for each group of rules, wherein the rule summary score of the group of rules is assigned to each clinical decision rule of the group of rules.

In another disclosed aspect, an electronic CDS method comprises: obtaining values of the preconditions of the clinical decision rules for a current patient, each clinical decision rule comprising a set of preconditions and being executable to generate a predicted value of a clinical conclusion that is dependent on values of the set of preconditions of the clinical decision rule; executing clinical decision rules using a computer to generate predicted values of clinical conclusions for the current patient based on the obtained values for the current patient of preconditions of the clinical decision rules; executing the clinical decision rules using the computer to generate predicted values of the clinical conclusions for past patients based on values for the past patients of the preconditions of the clinical decision rules retrieved from an EMR hosted by or connected with the computer; generating rule summary scores for the clinical decision rules using the computer based on comparisons of values of the clinical conclusions for the past patients retrieved from the EMR with the predicted values of the clinical conclusions for the past patients to prioritize the clinical decision rules or the predicted values generated by the clinical conclusion for the current patient; and displaying, on a display operatively connected with the computer, a ranking of the predicted values of the clinical conclusions as well as the corresponding rules for the current patient ranked at least in part by the rule summary scores of the clinical decision rules executed to generate the predicted values of clinical conclusions for the current patient.

One advantage resides in providing wider applicability of an electronic clinical decision support (CDS) device to diverse medical facilities.

Another advantage resides in an electronic CDS device providing clinical decision support that is better targeted to the served patient population.

Another advantage resides in providing an electronic CDS device employing clinical decision rules sets generated by multiple clinical studies or other multiple sources with improved harmonization between the diverse clinical decision rules sets.

Another advantage resides in providing an electronic CDS device having one or more of the foregoing advantages while employing vetted clinical decision rules that have been developed, validated, and approved by appropriate medical organizations, governmental agencies, and/or so forth.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention. Unless otherwise noted, the drawings are diagrammatic and are not to be construed as being to scale or to illustrate relative dimensions of different components.

FIG. 1 diagrammatically shows a CDS device and depicts a clinical decision rules ranking process performed by the CDS device.

FIG. 2 diagrammatically shows the CDS device of FIG. 1 and depicts a clinical decision support process performed by the CDS device to provide clinical decision support for a current patient in which the displayed clinical conclusions as well as the corresponding rules are ranked based at least in part on the rules ranking produced by the rules ranking process of FIG. 1.

FIG. 3 diagrammatically shows another CDS device embodiment that provides clinical decision rules ranking as disclosed herein.

### DETAILED DESCRIPTION

As described previously, the process for developing and validating clinical decision rules and for obtaining requisite approvals and/or certifications can be lengthy and costly, and as such is commonly carried out by large institutional entities. Moreover, it is commonly desired to construct an electronic CDS device with a breadth of applicability in terms of range the medical conditions covered, populations covered, and so forth, To this end, the electronic CDS device may employ clinical decision rules generated by different clinical studies performed on different patient populations that may vary widely in terms of demographics, general health, and the like. To remove potentially confounding variables, such clinical studies are sometimes intentionally restricted to certain demographic groups, e.g. being limited to only female subjects, or being limited to a particular age group, a particular ethnicity, and/or so forth. The resulting clinical decision rules may be reliably validated for patients meeting the strictures of the target population, but the validity of the rules to other patients may be questionable.

The accuracy of clinical decision rules for a given hospital or other given medical institution may also depend on the demographic characteristics of that hospital. For example, consider a rule that has 90% accuracy for the general population, but is more accurate than that for younger patients and less accurate for older patients. Such a rule applied in a hospital serving an older demographic may exhibit accuracy below 90% for that hospital; whereas, the rule applied in a hospital serving a younger demographic may exhibit accuracy above 90%. Such demographic dependencies may be more complex, e.g. a rule may be more (or less) accurate for a patient population having a certain set of demographic characteristics, and these may be difficult to define. For example, a rule may have different performance in a hospital serving patients drawn predominantly from a more affluent area, as compared with another hospital that serves patients drawn predominantly from a less affluent area. These dependencies are difficult to determine a priori.

Moreover, developing and validating clinical decision rules and obtaining appropriate approvals/certifications can be a lengthy and expensive process. Once validated and approved or certified, it may not be practical to modify a rule for a given hospital, as there may be no principled basis for overriding the extensive clinical studies and other underpinnings of the rule.

In sum, clinical decision rules in a knowledge base or integrated from multiple knowledge bases may represent very different contexts, for example, different clinical study periods, different patient populations, different evaluation criteria, and cohort designs. As a result, such clinical decision rules are not all applicable or informative for the subjects in a given healthcare setting, such as a particular hospital or a hospital network. For example, clinical decision rules can be developed based on a clinical study done 30 years ago on a small number of Caucasian patients, but then applied in year 2015 in a CDS device providing clinical decision support for a population of Chinese patients. Executing the rules from the various knowledge bases and feeding them without discrimination to care providers does not provide precise clinical decision support, but rather burdens the care providers with potentially inconsistent and confusing or even inaccurate supporting suggestions.

One way to address these difficulties is to limit a clinical decision support (CDS) device to employing clinical decision rules developed using clinical studies whose target populations closely match those of the deployment hospital. However, this approach greatly restricts the pool of clinical decision rules that may be incorporated into the CDS device. Moreover, an apparently close match between the study population and hospital population may nonetheless mask significant demographic differences between the two populations that may lead to accuracy of the clinical decision rule for the hospital population deviating significantly from the accuracy observed in the study population. Such a site-specific comparison of populations also can be costly, time consuming, and laborious.

Disclosed herein are improved CDS devices which leverage the huge amount of data generated daily in a typical hospital Electronic Medical Record (EMR), preferably a more specialized Clinical Data Repository (CDR; for conciseness we refer both to EMR in the following text), to tailor the CDS device to a target (e.g. hospital) population, without the need of annotations from a clinical study. The EMR stores the unique characteristics of the population, and in data driven approaches disclosed herein the underlying characteristics of the population as represented in the EMR provide prioritization of the clinical decision rules from multiple knowledge bases. In embodiments disclosed herein, the clinical decision rules are not discarded based on this tailoring rather, the clinical decision rules are prioritized on the basis of their accuracy for the hospital population as determined using the EMR data.

In one approach, determined values of preconditions of the clinical decision rules for past patients are retrieved from the EMR. The determined values of the clinical conclusions of the clinical decision rules for the past patients are also retrieved from the EMR. (The term “past” patient as used herein refers to patients whose medical records are stored in the EMR for which a clinical conclusion for a clinical decision rule has been determined and stored in the EMR. Such a “past” patient might possibly still be a patient at the hospital or may have since been re-admitted to the hospital the patient is a “past” patient in the sense that the clinical conclusion has been determined for the patient). These later conclusion values serve as “ground truth” information for the past patients. For each past patient, the clinical decision rules are executed using the values of the preconditions obtained for the past patient to generate predicted values of the clinical conclusions for the past patients. All predicted values of the past patients are collected. Rule summary scores for the clinical decision rules are generated based on comparisons of the retrieved determined values of the clinical conclusions for the past patients (i.e. the ground truth values) with the predicted values of the clinical conclusions for the past patients generated by the clinical decision rules. These rule summary scores indicate accuracy of the various clinical decision rules for the hospital population. In one embodiment, the rule summary scores are used, when providing clinical support for a patient currently under care (i.e. “current patient”), to rank the clinical conclusions produced by the EMR for the patient currently under care, so that care providers are presented with the most accurate clinical decision rules and the associated clinical conclusions for the patient currently under care, ranked highest (where accuracy is measured for the hospital population as just described). The approach ranks the existing clinical decision rules implemented by the CDS device on a record-based level, with the ranking based on the accuracy of each rule for the past patient population at the hospital or other medical institution where the CDS device is deployed. The clinical decision rules themselves are not altered, nor are the clinical conclusions drawn by those rules. In this way, the validated clinical decision rules are used in their intended manner, and the conclusions output by the clinical decision rules for a current patient are not modified in any way that might compromise the validity of the rules.

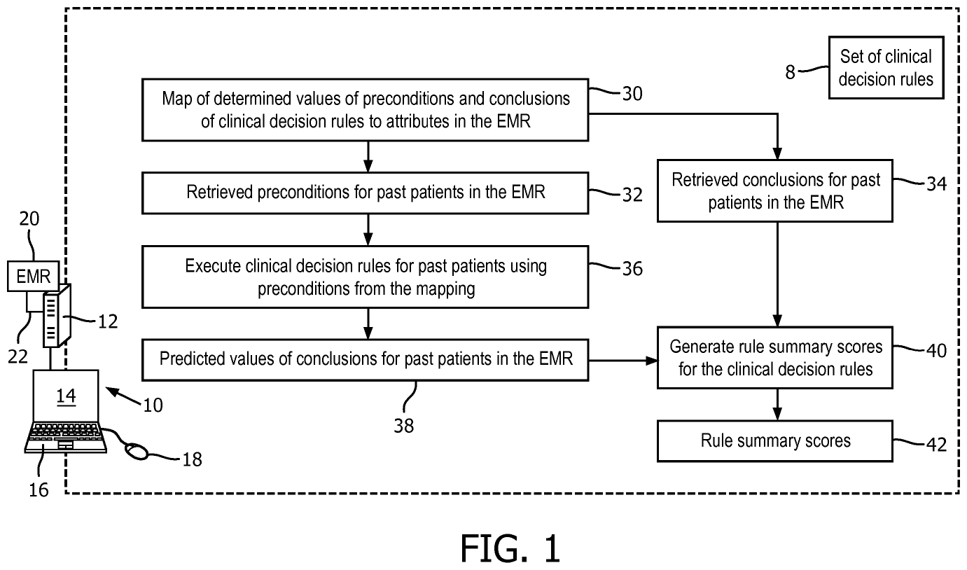

With reference to FIG. 1, a CDS device operates to apply a set of clinical decision rules 8 to provide clinical decision support for care workers. Each clinical decision rule of the set 8 generates one or more clinical conclusions if certain preconditions are met, and these are presented to the care worker by the CDS device. The set of clinical decision rules 8 may be drawn from various studies, each of which may in general be performed on a different study population having generally different demographics and/or other generally different population characteristics, e.g. the study populations may in general differ in terms of age distribution, gender distribution, geographical distribution, affluence distribution, and/or so forth. Approaches disclosed herein effectively harmonize biases introduced by these population differences by emphasizing those rules which are most accurate for the population of the hospital or other medical population being served by the CDS device.

The CDS device comprises one or more computers, e.g. an illustrative user computer 10 (e.g. a laptop computer, desktop computer, or so forth) networked with a server computer 12. The user computer 10 includes user interfacing components such as a display 14 and one or more user input devices, e.g. an illustrative keyboard 16 and mouse 18, and/or a touch-sensitive overlay of the display 14 (so that it is a touchscreen), or so forth. In the illustrative embodiment, it is assumed that the server computer 12 is a high computing capacity computer that executes the clinical decision rules 8, while the user computer 10 provides user interfacing to enable user inputs for using the CDS device, e.g. entry of a current patient identification for which clinical decision support is sought, and responsive display of clinical conclusions output by the server computer 12 executing the clinical decision rules for preconditions of the current patient. An electronic medical record (EMR) 20 resides on the server computer 12 (that is the server computer 12 hosts the EMR 20), or in other embodiments the EMR resides on (i.e. is hosted by) a different server computer networked with the server computer 12 by an electronic data network 22 (e.g. a hospital data network and/or the Internet or so forth) that provides CDS computational processing. In the illustrative embodiment, the user computer 10 may run a dedicated CDS device interface program for accessing the clinical decision rules execution engine of the server computer 12 or, alternatively, the user computer 10 may run a web browser that accesses the clinical decision rules execution engine residing on the server computer 12 via a hypertext transfer protocol (http) interface or the like. The server computer 12 may in some embodiments comprise a plurality of interconnected servers forming a cloud computing resource. These are merely illustrative arrangements, and other configurations are contemplated, e.g. a single computer may perform both clinical decision rules execution processing and user interfacing operations.

The Electronic Medical Record (EMR) 20 is to be understood as encompassing any electronic medical record storing past and current patient data (i.e. attributes) and networked with or otherwise connected to be read by the CDS device. The EMR 20 may be known by other nomenclatures, e.g. an Electronic Health Record (EHR) or a Clinical Data Repository (CDR), and/or may be configured as two or more different electronic databases, e.g. a general-purpose electronic medical record and one or more specialized electronic medical records such as a Picture Archive and Communication System (PACS) specialized for medical imaging medical recordation, and/or a cardiovascular information system (CVIS) specialized for medical recordation of cardiovascular-centric patient information, and/or so forth. The term “Electronic Medical Record” or “EMR” as used herein is intended to encompass all such database(s) that store past and current patient information (i.e. attributes) of relevance to the clinical decision rules 8 of the CDS device.

Not shown in FIG. 1 is a non-transitory storage medium storing instructions readable and executable by the one or more computers 10, 12 to perform the disclosed clinical decision support operations. The non-transitory storage medium may, for example, comprise one or more of a hard disk drive or other magnetic storage medium, an optical disk or other optical storage medium, a solid state drive, flash memory or other electronic storage medium, various combinations thereof, or so forth. In general, the instructions include stored instructions for executing the set of clinical decision rules 8. A clinical decision rule is comprised of the preconditions (e.g. the “if” part) and clinical conclusions (e.g. the “then” part). An example clinical decision rule is as follows: If A is a, B is b, then C is c. If a clinical decision rule is in the form of a risk assessment score, it can be in the form as follows: If A is a, B is b, then the risk score of C is s. As a result, the rule defined here covers risk scoring which is a specialized form of clinical decision rules. More formally, a clinical decision rule can be heuristically written as:
If <preconditions met by patient> then present <clinical conclusion>
   For a given patient, the clinical decision rule is executed by the server computer 12 using the values of the preconditions for that patient retrieved from the EMR 20 to generate the value of the clinical conclusion. The instructions further include instructions, e.g. executed by the user computer 10, to enable a care giver to identify a patient for whom clinical decision support is sought, for example by entering the patient's social security number, patient identifier (PID), or other identifying information via a user input device 16, 18, and to display, on the display 14, the clinical conclusions generated by executing the clinical decision rules 8 with the preconditions for the patient retrieved from the EMR 20.

FIG. 1 diagrammatically illustrates further operations performed by the CDS computer 10, 12 executing the stored instructions. These operations perform rules summary scoring to assess the accuracy of the clinical decision rules 8 for patients at the particular hospital or medical institution. In the illustrative example, the set of patients for which the rules summary scoring is performed is the set of past patients stored in the EMR 20 for whom the EMR 20 stores values for both the preconditions and the clinical conclusions. These stored values of the clinical conclusions provide “ground truth” values for these conclusions against which the predictions produced by the clinical decision rules 8 can be compared to assess prediction accuracy. The rules summary scoring method employs a mapping 30 between of determined values for preconditions and clinical conclusions of the clinical decision rules 8 to attributes in the EMR 20. This mapping 30 may be provided manually, e.g. using a manually created relational database, table, or other data structure storing links between rule preconditions and clinical conclusions on the one hand, and data fields of the EMR 20 on the other hand. Alternatively, the mapping 30 may be automatically generated if the EMR 20 employs a standard structure, searchable clinical terms, or the like so as to enable the relevant data fields of the EMR 20 to be automatically identified.

Using the mapping 30, the preconditions 32 and clinical conclusions 34 stored for past patients are retrieved from the EMR 20. The clinical conclusions 34 serve as “ground truth” values for these conclusions, as they are conclusions that have been drawn by medical professionals on various presumed reliable bases, e.g. medical tests, exploratory surgeries, medical imaging, physical examination by medical professionals, or so forth, and deemed sufficiently reliable to be recorded in the patient's electronic medical record. In some cases the clinical conclusion stored in the EMR 20 was arrived at in due course as the patient's disease or other medical condition progressed to a point where the clinical conclusion manifested as readily interpreted observable symptoms. The retrieved preconditions 32 enable performing an operation 36 in which the clinical decision rules 8 are executed for the past patients using the retrieved preconditions 32 so as to generate predicted values 38 for the clinical conclusions for the past patients in the EMR 20. In an operation 40, these predicted values 38 for the clinical conclusions are compared with the “ground truth” clinical conclusions 34 retrieved from the EMR 20, and these comparisons are used to assess accuracy (in a statistical sense) of each clinical decision rule for the past patients whose data (including clinical conclusions) are stored in the EMR 20. These comparisons are stored as rule summary scores 42, and provide empirical metrics of the accuracy of each clinical decision rule for patients of the target hospital (as represented by the past patients whose data are stored in the EMR 20).

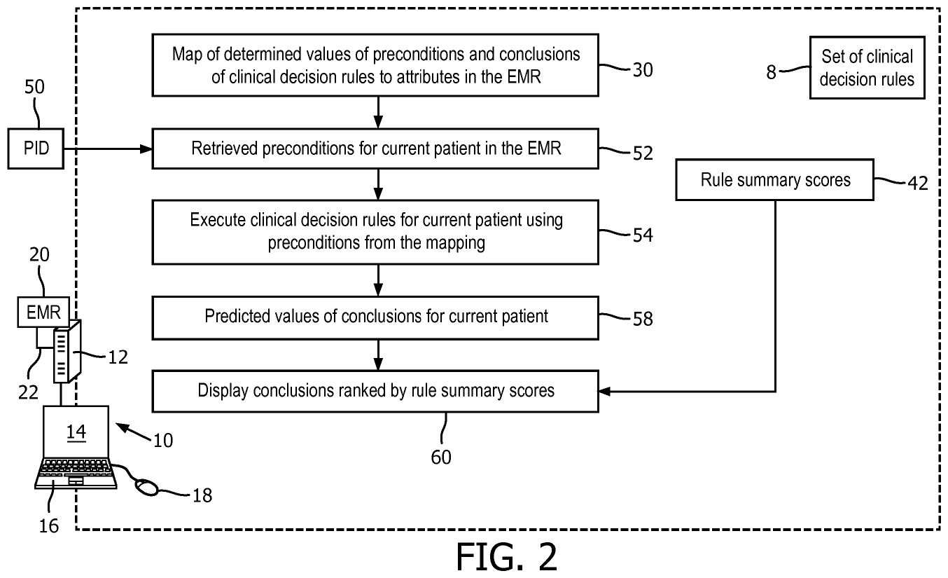

With reference now to FIG. 2, further operations performed by the CDS computer 10, 12 executing the stored instructions are diagrammatically illustrated. These operations perform clinical decision support for a current patient suitably identified by a patient identifier (PID) 50 or other patient-identifying information. In an operation 52, the mapping 30 already described with reference to FIG. 1 is used to retrieve determined values for preconditions of the clinical decision rules 8 from the EMR 20. (Note that for a current patient, values for the clinical conclusions are generally not yet available, at least as pertains to clinical decisions for which support is sought by care givers.) Alternatively, the user computer 12 may be programmed to obtain one, two, more, or all values of the preconditions of the clinical decision rules 52 for the current patient by receiving the values for the current patient via the one or more user input devices 16, 18.

In an operation 54, the clinical decision rules 8 are executed for the current patient using the retrieved preconditions 52 so as to generate predicted values 58 for the clinical conclusions for the current patient. In an operation 60, these predicted values 58 for the clinical conclusions are displayed on the display 14. In the operation 60, the clinical conclusions are organized in accordance with the rule summary scores 42 in a way that highlights or draws most attention to those clinical conclusions that are most accurate for the hospital population as indicated by the rule summary scores 42. In one approach, the clinical conclusions are ordered by the rule summary scores 42, with the clinical conclusions having highest rule summary scores listed first together with the rules applied and the clinical conclusions having lowest rule summary scores listed last. In other embodiments, only a “top N” clinical conclusions and rules are listed, e.g. the clinical conclusions of the N rules having highest rule summary scores are displayed on the display 14, with the care giver provided with a user interfacing option (e.g. a scroll bar) by which the care giver can scroll down to clinical conclusions generated by clinical decision rules with lower rule summary scores.

In another variant embodiment, the clinical decision rules are organized in accordance with the rule summary scores 42 and the clinical decision rules are displayed to the care giver in accord with this organization (e.g. ordered by rule summary scores 42). The care giver can then select the clinical decision rules to be executed, and only the selected clinical decision rules are executed. This approach can reduce total processing time by executing only those clinical decision rules identified by the care giver.

Optionally, the rule summary scores may be displayed along with the clinical conclusions and rules in an intuitive fashion. For example, those clinical decision rules having rule summary scores above a high reliability threshold Tx are designated as highly reliable rules. Those clinical decision rules having rule summary scores below a low reliability threshold TL are designated as low reliability rules. The clinical conclusions may then be flagged on the display 14 based on the reliability of the generating rules. For example, clinical conclusions generated by low reliability clinical decision rules may be highlighted in yellow, italicized, or otherwise indicated to be of questionable reliability. Optionally, clinical conclusions generated by high reliability clinical decision rules may be highlighted in red, boldface, or otherwise indicated to be of high reliability.

The skilled artisan will, upon reading the foregoing and this disclosure in full, appreciate the benefit of this disclosed approach. Advantageously, the care giver is provided with all clinical conclusions generated by the CDS device without any modification of those conclusions; yet, the clinical conclusions are presented in a way that ensures the most accurate conclusions (in a statistical sense, as measured by the rule summary scores 42) are given most prominence. As the clinical conclusions are not modified, any properties of the clinical decision rules 8 such as validation, regulatory approval, certification by clinical organizations, or so forth remain intact. On the other hand, clinical conclusions of highest reliability for the population served by the hospital are emphasized to care givers, while clinical conclusions of lower reliability are de-emphasized or optionally highlighted as potentially unreliable.

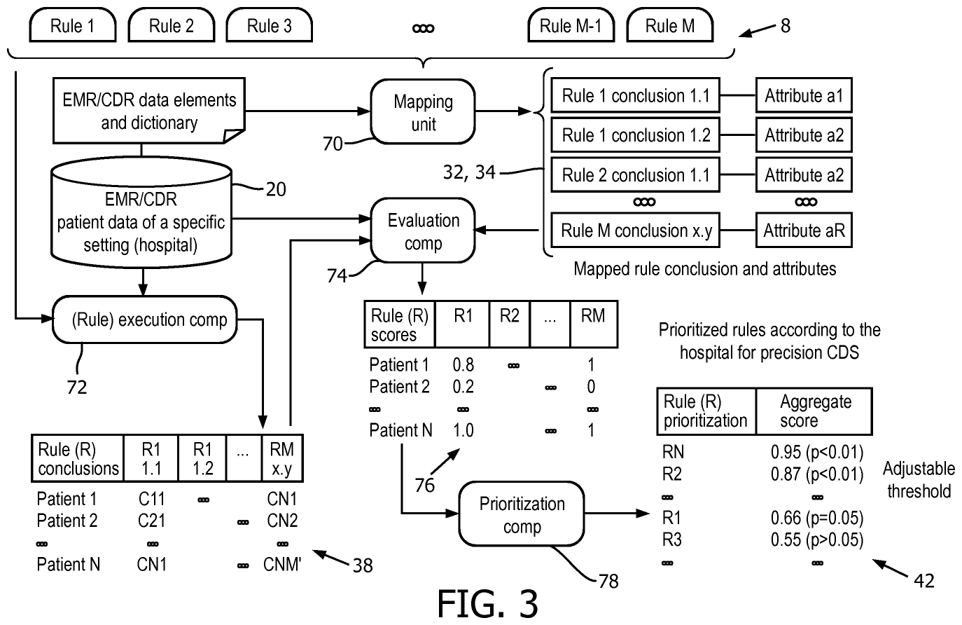

With reference now to FIG. 3, another illustrative embodiment is described of the disclosed automatic data driven approach to prioritize relevant clinical decision rules according to a specific healthcare setting to achieve precision knowledge utilization from multiple bases. In describing FIG. 3, like reference numbers to those of FIGS. 1 and 2 are used where components of the embodiment of FIG. 3 correspond with components of FIGS. 1 and 2. The CDS device of FIG. 3 includes a mapping unit 70 that maps preconditions 32 and rule conclusions 34 to the attributes of patient data from the EMR 20 (optionally including a differently named clinical data repository, i.e. CDR, in this example). An auto-execution component 72 runs all executable preconditions of various clinical decision rules on past patient data, and stores the rule outputs (clinical conclusions) for the whole past patient population of the corresponding healthcare setting, so as to produce the predicted values 38 of the clinical conclusions. An evaluation component 74 compares the predictions 38 for the clinical conclusions of the clinical decision rules 8 with the clinical conclusions 34 obtained from the EMR 20 to generate a tabulation 76 of characteristic (consistency) score for each clinical decision rule and for each patient.

A prioritization component 78 then ranks the clinical decision rules 8 according to the evaluation scores 78 across multiple patients and multiple consistency (corresponding to the operation 40 of the embodiment of FIG. 1), optionally with a threshold to control the prioritization stringency, so as to produce the rule summary scores 42. Although not explicitly shown in FIG. 3, it will be appreciated that the various computational components 70, 72, 74, 78 of the CDS device embodiment of FIG. 3 may be performed by the computer 10, 12 executing instructions stored on the aforementioned non-transitory storage medium.

In the following, some further examples are given, using the general framework described above with reference to FIG. 3.

The set of clinical decision rules 8 can contain multiple rules, and an integrated knowledge base can be optionally created by consolidating clinical decision rules from various knowledge bases and converting them into the unified format under consistent concepts. Suppose this results in M rules: Rule 1, Rule 2, . . . , Rule M, where multiple rules can be from the same knowledge base, e.g. Rule 1 and Rule 2 from Knowledge base 1, and Rule 3 from Knowledge base 2, so on and so forth.

In a specific healthcare setting (e.g. a hospital or a hospital network), a database of patient data is referred to herein as the Electronic Medical Record (EMR) 20 but which may in general be variously embodied and/or named, e.g. a clinical data repository (CDR). With the large amount of patient data in daily practice, the EMR 20 stores attributes (data columns) that encompass diverse clinically relevant information. The CDS devices disclosed herein recognize that the EMR 20 can reveal the unique characteristics of the patient population under the specific healthcare setting. Suppose there are r attributes a1, a2, . . . , aR.

The mapping unit 70 maps the rule preconditions 32 and conclusions 34 with the attributes stored in the EMR 20 into matched pairs, e.g. Rule 1 conclusion 1.1 (abbreviation R1 1.1)—attribute a1, Rule 1 conclusion 1.2—attribute a2, Rule 2 conclusion 2.1—attribute 2, . . . . Similarly, the rule preconditions can be also mapped to the EMR 20 attributes, e.g. A-a3, B-a4, . . . . This mapping enables the proper linkage between the clinical decision rules and the EMR 20 data elements and dictionary. In this way the preconditions of a clinical decision rule can be executed on a patient given his/her attribute values in the EMR 20, and accordingly the rule conclusion (e.g. R1 1.1) can be also compared with the matched attribute (e.g. a1).

To perform this comparison on the whole (past) patient population, the execution component 72 on each rule retrieves the attribute values matching the preconditions, and collects the conclusion value(s), as illustrated in Table 1, where C11, C21, . . . , CN1 denote the Rule 1 conclusion 1.1 values for patients 1, 2, . . . , N.

TABLE 1

| Rule (R) Conclusions | R1 1.1 | R1 1.2 | . . . | RM x.y |
|---|---|---|---|---|
| Patient 1 | C11 | . . . | | CN1 |
| Patient 2 | C21 | | . . . | CN2 |
| . . . | . . . | | | . . . |
| Patient N | CN1 | | . . . | CNM' |

The evaluation component 74 compares all these executed rule conclusion values (C**) with the mapped attribute values (a1, a2, . . . ) on the whole past patient population. A score is used on a per-past patient and per-rule basis to measure the consistency between the rule conclusions and the patient characteristics from EMR 20. In one embodiment, this score can be as follows:

scrᵢⱼ=0 if cᵢⱼ!=a_{ij′}, and scrᵢⱼ=1 if cᵢⱼ==a_{ij′}

where c_{ij′} is the conclusion for patient i and clinical conclusion (i.e. column) j, and a_{ij′} is the value of patient i for attribute j′ (i.e. the “ground truth” clinical conclusion retrieved from the EMR 20), and j-j′ represents the mapped rule conclusion j and attribute j′.

In other embodiments, the score can be based on more complex scoring schemes and/or external references. In some such embodiments, weights are introduced to score the knowledge bases, based on the authority rankings, and/or similarity scores of the clinical studies, based on the ethnic groups, data size, guideline relevance, etc. A normalization step can be further applied to scale each scrᵢⱼ into [0, 1].

In general, for one clinical decision rule there can be multiple clinical conclusions (e.g. R1 1.1, R1 1.2 for Rule 1), in one embodiment, a normalization score can be implemented to further aggregate multiple scores belonging to one rule into one, such that each rule can have a concise evaluation and rules can be compared accordingly. After the aggregation, the rule scores 76, one for each rule and each patient, can be illustrated as (by way of non-limiting illustration) in Table 2.

TABLE 2

| Rule (R) Scores | R1 | R2 | . . . | RM |
|---|---|---|---|---|
| Patient 1 | 0.8 | . . . | | 1 |
| Patient 2 | 0.2 | | . . . | 0 |
| . . . | . . . | | | . . . |
| Patient N | 1.0 | | . . . | 1 |

In the tabulation 76 of the rule consistency scores for the past patients (e.g. Table 2), the rule consistency scores for each past patient comprise comparisons of the retrieved determined values of one or more clinical conclusions of each rule for the past patient with the predicted values of the one or more clinical conclusions for the past patient.

With the overall evaluation scores 76 available, the prioritization component 76 sorts the clinical decision rules 8 in accordance with rule summary scores 42 (where FIG. 3 shows the clinical decision rules ranked by their listed rule summary scores 42). This ranking is thus according to the characteristics of the specific healthcare setting (e.g. hospital or hospital network) as reflected in the EMR 20.

In embodiments employing an online mode (e.g. accessed via a web interface) or otherwise calling for efficient computation, the rule summary score can be generated for each clinical decision rule on the whole population, and the prioritization is simply the ranking of all clinical decision rules with respect to their rule summary scores. In one embodiment, the rule summary score Sᵢ for rule i is:

$${S}_{i} = {\sum }_{j} {s}_{ij} \text{/} N$$

where the summation on j is over all N past patients, and so N is used as the normalization factor in the above-expressed rule summary score.

In another embodiment, the data quality and completeness can be included. Suppose for patient j (one row in the tabular representation of the EMR 20), the ratio of non-missing and non-outlier can be denoted as rⱼ, then the rule summary score for rule i can be further proposed as:

$${S}_{i} = {\sum }_{j} {s}_{ij} {r}_{j} \text{/} N$$

The previous summary score

$${S}_{i} = {\sum }_{j} {s}_{ij} \text{/} N$$

is a special case of this generalized score

$${S}_{i} = {\sum }_{j} {s}_{ij} {r}_{j} \text{/} N$$

for an ideal setting where all data is fully clean and complete (so rⱼ=1 for every j).

The foregoing are merely illustrative examples, and other rule summary score formulations are also contemplated. In general, the rule summary score formulation is chosen to effectively measure the overall matching and consistency of the past patient data (patient by patient other than precondition by precondition without considering individual effects) to the clinical conclusion predictions produced by the clinical decision rules, disregarding the rule differences across individual patients.

In some further embodiments, a different prioritization approach is adopted for generating the rule summary scores 42. In the previous embodiments, the import of two clinical decision rules having the same rule summary score is that both rules have the same overall consistency on the past patient data. However, the two clinical decision rules may match different proportions of patients. To better model the consistency up to the individual level, a clustering algorithm can be applied on the full elements of the evaluation score table 76. Some suitable clustering algorithms include (as non-limiting illustrative examples) k-means clustering or hierarchical clustering, with L1 or L2 norm as the distance metric. After clustering, similar clinical decision rules (columns of the evaluation score table 76) in terms of the scores across rows (across patients) of the table are close to each other, and dissimilar rules are far away from each other in the grouping. An illustration of the clustered results is shown in Table 3.

TABLE 3

| Clustered | | | | | | | |
|---|---|---|---|---|---|---|---|
| Rules (R) | R1 | R6 | R9 | . . . | R8 | R9 | RM |
| Patient 1 | 0.89 | 0.89 | 0.90 | | 0.1 | 0.09 | 0.09 |
| Patient 2 | 0.20 | 0.21 | 0.20 | . . . | 0.98 | 1.0 | 1.0 |
| . . . | . . . | . . . | . . . | | | | . . . |
| Patient N | 1.0 | 1.0 | 1.0 | . . . | 0.49 | 0.49 | 0.5 |

As shown in Table 3, the cluster containing R1, R6, and R9 show high consistency scores for patients 1 and N but not patient 2. On the contrast, another illustrative cluster containing R8, R9 and RM shows high consistency for patient 2, medium consistency for patient N and low for patient 1. The rules within one cluster are similar across the rows while they are dissimilar with rules from the other clusters. In some embodiments, the clustering employs a similarity metric measuring per-past patient similarity of the comparisons of the retrieved values of the clinical conclusions for the past patients with the predicted values of the clinical conclusions for the past patients.

With the resultant clusters, a cluster summary score can be obtained for each cluster, and then the top 1 or multiple clusters can be selected, and thus the prioritized rules belonging to them are obtained as the final outputs (e.g., with the rule summary scores assigned in accordance with the clusters to which they belong). A cluster summary score can adopt the summary score embodiment (average of all cᵢⱼ in the cluster, e.g. all light orange cells averaged for the illustrative table above), and more sophisticated variant embodiments can be also adopted. In this clustering approach to ranking the clinical decision rules 8, the rule summary score of each group of rules is assigned to each clinical decision rule of the group of rules. In this way, the rule summary scores 42 operate to rank the different groups of rules while keeping each group of rules together.

In some embodiments, an adjustable threshold can be introduced to permit the user to distinguish informative rules from less precise rules for specific clinical situations. As illustrated in Table 4, in one embodiment, a p-value threshold is introduced to prioritize clinical decision rules according to their statistical significance (a smaller p-value indicates a more statistically significant result). For example, clinical decision rules with p-values >0.05 (e.g. R3 and rules below in the ranked list) are less significant in Table 4.

TABLE 4

| Rule (R) Prioritization | Aggregate score |
|---|---|
| RN | 0.95 (p < 0.01) |
| R2 | 0.87 (p < 0.01) |
| . . . | . . . |
| R1 | 0.66 (p = 0.05) |
| R3 | 0.55 (p > 0.05) |
| . . . | . . . |

In some embodiments, special handling is provided for any “non-starter” rules. Non-starter rules, as used herein, are those rules that do not have sufficient mapped preconditions and/or clinical conclusions in the past patient data stored in the EMR 20. As a result, there are no evaluation scores for these rules in the table 76. Such non-starter clinical decision rules could be down-scored to 0, but doing so might omit potentially useful CDS information. Therefore, in some embodiments the non-starter clinical decision rules are moved up to be just above the threshold in order not to lose any potentially useful rules.

To calculate the p-value for one rule, a statistical test can be adopted. In one embodiment, a Chi-square test is employed. For a rule, it can provide a conclusion with multiple values (Yes/No, or <=/>= a certain threshold). For the mapped attribute in the EMR 20, there are also multiple values accordingly. A contingency table across the rule conclusion and the mapped attribute values on the full patient data can be constructed, and then the p-value of the Chi-square test can be calculated accordingly. This is merely an illustrative example, and other statistical tests can be employed.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. An electronic clinical decision support (CDS) device comprising:
a database storing clinical decision rules, each clinical decision rule comprising a set of preconditions and being executable to generate a predicted value of a clinical conclusion that is dependent on values of the set of preconditions of the clinical decision rule;
a computer hosting or connected by a data network with an electronic medical record (EMR) of a past patient, the EMR containing determined values of preconditions and determined values of a clinical conclusion, the computer programmed to perform clinical decision support for a current patient by obtaining values of the preconditions of the clinical decision rules for the current patient and executing the clinical decision rules using the obtained values of the preconditions for the current patient to generate predicted values of the clinical conclusions for the current patient, the computer further programmed to perform a rules ranking process including:
retrieving, from the EMR, the determined values of the preconditions of the clinical decision rules for past patients and the determined values of the clinical conclusions of the clinical decision rules for the past patients;
for each past patient, executing the clinical decision rules using the values of the preconditions obtained for the past patient to generate predicted values of the clinical conclusions for the past patients; and
generating rule summary scores for the clinical decision rules based on comparisons of the retrieved determined values of the clinical conclusions for the past patients with the predicted values of the clinical conclusions for the past patients to prioritize the clinical decision rules or the predicted values generated by the clinical conclusion for the current patient.

2. The electronic CDS device of claim 1 wherein generating the rule summary scores for the clinical decision rules includes:
clustering clinical decision rules into groups of rules using a similarity metric; and
generating a rule summary score for each group of rules wherein the rule summary score of the group of rules is assigned to each clinical decision rule of the group of rules.

3. The electronic CDS device of claim 2 wherein the clustering operates on a tabulation of rule consistency scores for the past patients where the rule consistency scores for each past patient comprise comparisons of the retrieved determined values of one or more clinical conclusions of each rule for the past patient with the predicted values of the one or more clinical conclusions for the past patient.

4. The electronic CDS device of claim 1 wherein generating the rule summary scores for the clinical decision rules comprises:
computing the rule summary score Sᵢ for clinical decision rule i as:
$${S}_{i} = {\sum }_{j = 1}^{N} \frac{{s}_{ij}}{N}$$
where N is the number of past patients for which clinical decision rule i is executed and sᵢⱼ is a quantitative comparison of the retrieved value of the clinical conclusion of clinical decision rule i for a past patient j with the predicted value of the clinical conclusion of clinical decision rule i for the past patient j.

5. The electronic CDS device of claim 1 wherein generating the rule summary scores for the clinical decision rules comprises:
computing the rule summary score Sᵢ for clinical decision rule i as:
$${S}_{i} = {\sum }_{j = 1}^{N} \frac{{s}_{ij} {r}_{j}}{N}$$
where N is the number of past patients for which clinical decision rule i is executed and sᵢⱼ is a quantitative comparison of the retrieved value of the clinical conclusion of clinical decision rule i for a past patient j with the predicted value of the clinical conclusion of clinical decision rule i for the past patient j and rⱼ is a data reliability metric.

6. The electronic CDS device of claim 4 wherein:
each clinical decision rule of the set of clinical decision rules is executable to generate a binary predicted value;
the quantitative comparison sᵢⱼ has value sᵢⱼ=1 if the retrieved value of the clinical conclusion of clinical decision rule i for a past patient j is the same as the predicted value of the clinical conclusion of clinical decision rule i for the past patient j; and
the quantitative comparison sᵢⱼ has value sᵢⱼ=0 if the retrieved value of the clinical conclusion of clinical decision rule i for a past patient j is not the same as the predicted value of the clinical conclusion of clinical decision rule i for the past patient j.

7. The electronic CDS device of claim 1 further comprising:
a display connected with the computer and configured to display at least a sub-set of the predicted values of clinical conclusions and the corresponding clinical decision rules applied for the current patient ranked at least in part by the rule summary scores.

8. The electronic CDS device of claim 7 wherein:
each clinical decision rule is executable to generate a binary predicted value of a clinical conclusion predicting whether the clinical conclusion holds; and
the display is configured to display the clinical conclusions predicted to hold for the current patient ranked at least in part by the rule summary scores of the clinical decision rules executed to generate the predicted values of clinical conclusions for the current patient.

9. The electronic CDS device of claim 1 further comprising:
a display, wherein the display is configured to display at least a sub-set of the predicted values of clinical conclusions and the corresponding clinical decision rules applied for the current patient and to include an indication of any predicted values of clinical conclusions for the current patient that are produced by clinical decision rules whose rule summary scores indicate reliability of the clinical decision rule is below a threshold reliability.

10. The electronic CDS device of claim 1 further comprising:
one or more user input devices;
wherein the computer is programmed to obtain values of the preconditions of the clinical decision rules for the current patient by at least one of retrieving the values for the patient from the EMR and receiving the values for the current patient via the one or more user input devices.

11. The electronic CDS device of claim 1 wherein the rules ranking process further includes:
mapping data fields of the EMR to the preconditions and clinical conclusions of the clinical decision rules;
wherein the retrieving from the EMIR of values of the preconditions of the clinical decision rules for past patients and values of the clinical conclusions of the clinical decision rules for the past patients is performed using the mapping (30) of data fields of the EMR to the preconditions and clinical conclusions of the clinical decision rules.

12. A non-transitory storage medium storing:
a database storing clinical decision rules, each clinical decision rule comprising a set of preconditions and being executable to generate a predicted value of a clinical conclusion that is dependent on values of the set of preconditions of the clinical decision rule; and
instructions readable and executable by a computer to perform an electronic clinical decision support (CDS) method including:
obtaining values of the preconditions of the clinical decision rules for a current patient;
executing the clinical decision rules to generate predicted values of clinical conclusions for the current patient based on the obtained values for the current patient of preconditions of the clinical decision rules;
retrieving, from an Electronic Medical Record (EMR), values of the preconditions of the clinical decision rules for past patients and values of the clinical conclusions of the clinical decision rules for the past patients;
for each past patient, executing the clinical decision rules using the values of the preconditions retrieved from the EMIR for the past patient to generate predicted values of the clinical conclusions for the past patients; and
generating rule summary scores for the clinical decision rules based on comparisons of the retrieved values of the clinical conclusions for the past patients with the predicted values of the clinical conclusions for the past patients to prioritize the clinical decision rules or the predicted values generated by the clinical conclusion for the current patient.

13. The non-transitory storage medium of claim 12 wherein generating the rule summary scores includes:
clustering the clinical decision rules into groups of rules; and
generating a rule summary score for each group of rules wherein the rule summary score of the group of rules is assigned to each clinical decision rule of the group of rules.

14. The non-transitory storage medium of claim 12 further comprising, displaying on a display a ranking of predicted values of clinical conclusions and the corresponding clinical decision rules applied for the current patient ranked at least in part by the rule summary scores of the clinical decision rules executed to generate the predicted values of clinical conclusions for the current patient.

15. An electronic clinical decision support (CDS) method comprising:
obtaining values of the preconditions of the clinical decision rules for a current patient, each clinical decision rule comprising a set of preconditions and being executable to generate a predicted value of a clinical conclusion that is dependent on values of the set of preconditions of the clinical decision rule;
executing clinical decision rules using a computer to generate predicted values of clinical conclusions for the current patient based on the obtained values for the current patient of preconditions of the clinical decision rules;
executing the clinical decision rules using the computer to generate predicted values of the clinical conclusions for past patients based on values for the past patients of the preconditions of the clinical decision rules retrieved from an Electronic Medical Record (EMR) hosted by or connected with the computer and;
generating rule summary scores for the clinical decision rules using the computer based on comparisons of values of the clinical conclusions for the past patients retrieved from the EMR with the predicted values of the clinical conclusions for the past patients to prioritize the clinical decision rules or the predicted values generated by the clinical conclusion for the current patient.
